(19) United States
(12) Patent Application Publication
(10) Pub. No.: US 2008/0200974 A1
(43) Pub. Date: Aug. 21, 2008

(54) Drug Eluting Stent System with Controlled Self Expansion

(75) Inventors: Brett A. Trauthen, Newport Beach, CA (US); Stefan Verheye, Hove (BE)

Correspondence Address: Crockett & Crockett, Suite 400, 24012 Calle De La Plata, Laguna Hills, CA, 92653, US

(73) Assignee: Cardiac Innovations, LLC

(21) Appl. No.: 11/675,451

(22) Filed: Feb. 15, 2007

(51) Int. Cl. A61F 2/06

(52) U.S. Cl. 623/1.2

(57) ABSTRACT

A self-expanding stent providing a high compliance mismatch upon initial placement, to exert radially outward force over the course of several weeks or months until it reaches the limits of expansion as imposed by a limiting mechanism, and thereafter provide high resistance to inward radial forces exerted by the artery without exerting additional outward radial force on the artery. The stent is coated with a compound including an MMPI, in a formulation in which the MMPI is substantially exhausted over the course of several weeks or months, to be substantially coincident with expansion of the stent to its stop limited diameter.

### FIELD OF THE INVENTIONS

The inventions described below relate the field of drug eluting stents.

### BACKGROUND OF THE INVENTIONS

Stents are now commonly used to treat blockages of coronary arteries. Although stents have proven to be effective at treating coronary artery occlusion, in early clinical use the coronary arteries, even after stenting, often became occluded through a process referred to as restenosis. Recently, stents coated with drugs which inhibit restenosis have been adopted, and have proven somewhat successful in maintaining long term patency of the stent and the artery. Compounds which inhibit excessive growth of arterial tissue in the vicinity of the stent are applied to the stent, and may be directly applied or incorporated into a polymer substrate that is applied to the stent. Currently, compounds such as sirolimus (rapamycin) and Taxol® (paclitaxel) are used as anti-restenosis agents in commercially available stents. Though these drug-eluting stents are proving effective at limiting restenosis, the additional problem of late mal-opposition remains to be solved. Malapposition refers to gaps between the wall of the coronary artery surrounding the stent and the stent itself (i.e., separation of the stent struts from the intimal surface of the arterial wall). Malapposition appears to be the result of the retreat of the native blood vessel from the stent and inability of the stent to further expand after placement, such that a balloon-expandable stent placed in a blood vessel will eventually cause the blood vessel to retreat away from the stent, leaving the stent loose or disengaged from blood vessel wall. Malapposition occurs in approximately 10 to 20% of drug eluting stent placements (Serruys, et al., Intravascular Ultrasound Findings In The Multicenter, Randomized, Double-Blind RAVEL (Randomized study with the sirolimus-eluting VElocity balloon-expandable stent in the treatment of patients with de novo native coronary artery Lesions) Trial, 106 Circulation 798 (2002)), whereas the incidence of malapposition of uncoated stents is only about 5% (Hong, et al., Incidence, Mechanism, Predictors, and Long-Term Prognosis of Late Stent Malapposition After Bare-Metal Stent Implantation, 109 Circulation 881 (2004)). This may lead to various problems, especially thrombosis and embolism. See, for example, Takano, et al., Late Coronary Thrombosis In A Sirolimus-Eluting Stent Due To The Lack Of Neo-intimal Coverage, 27 European Heart Journal 1133 (2006).

Several patents discuss coating of stents with metalloproteinase inhibitors (MMPI's) as a means of inhibiting restenosis. Recent published patent applications propose numerous MMPI compounds for stent coatings, including U.S. Patent Publications 20060036126, Caravatti, Carrier For Releasing A Therapeutic Substance In Response To The Presence Of An Enzyme, U.S. Pub. 20060035897 (Feb. 16, 2006), Hossainy, Carrier For Releasing A Therapeutic Substance In Response To The Presence Of An Enzyme, U.S. Pub. 20060009840 (Jan. 12, 2006), 20050209244, 20050203610 and 20050095267. Prescott, Organic Compounds, U.S. Pub. 20060035879 (Feb. 16, 2006), however, proposes use of an MMPI to promote endothelial re-growth, in seeming contradiction to proposal of Carvatti and Hossainy to use MMPI's to limit restenosis.

Regarding mechanisms for limiting expansion of stents, Duerig, et al., Composite Self Expanding Stent Device Having A Restraining Element, U.S. Pat. No. 6,179,878 (Jan. 30, 2001) discloses a self-expanding stent wrapped in a plastically deformable hoop or coil which limits self-expansion of the stent to avoid harm to the blood vessel in which it is placed. Pacetti, Self-expanding Stent with Variable Radial Force, U.S. Pat. No. 6,663,664 (Dec. 16, 2003) discloses a self-expanding stent wrapped in a biodegradable coil intended to control the expansion of the stent but eventually allow complete expansion (limited only by the force exerted by the blood vessel itself).

### SUMMARY

The methods and devices described below provide for coincident exhaustion of the therapeutic agent on a stent with the expansion of the stent to an expanded state as limited by a limiting mechanism. The stent is manufactured to provide a high compliance mismatch upon initial placement, exert radially outward force over the course of several weeks or months until it reaches the limits of expansion as imposed by the limiting mechanism, and thereafter provide high resistance to inward radial forces exerted by the artery without exerting additional outward radial force on the artery. The stent is coated with a compound including an MMPI, in a formulation in which the MMPI is substantially exhausted over the course of several weeks or months. Anti-proliferative agents may also be applied to the stent. The goal of the method is to use the expansive force of the self-expanding stent to force the vessel open while the MMPI is eluting, and then stop the stent induced expansion at about the same time that the MMPI is depleted.

### BRIEF DESCRIPTION OF THE DRAWINGS

FIGS. 1a, 1b and 1c illustrate a stent for use in implementing the therapeutic regimen.

FIGS. 2a, 2b and 2c illustrate a stent for use in implementing the therapeutic regimen.

FIG. 3 illustrates the therapeutic regimen to be achieved by the stent.

### DETAILED DESCRIPTION OF THE INVENTIONS

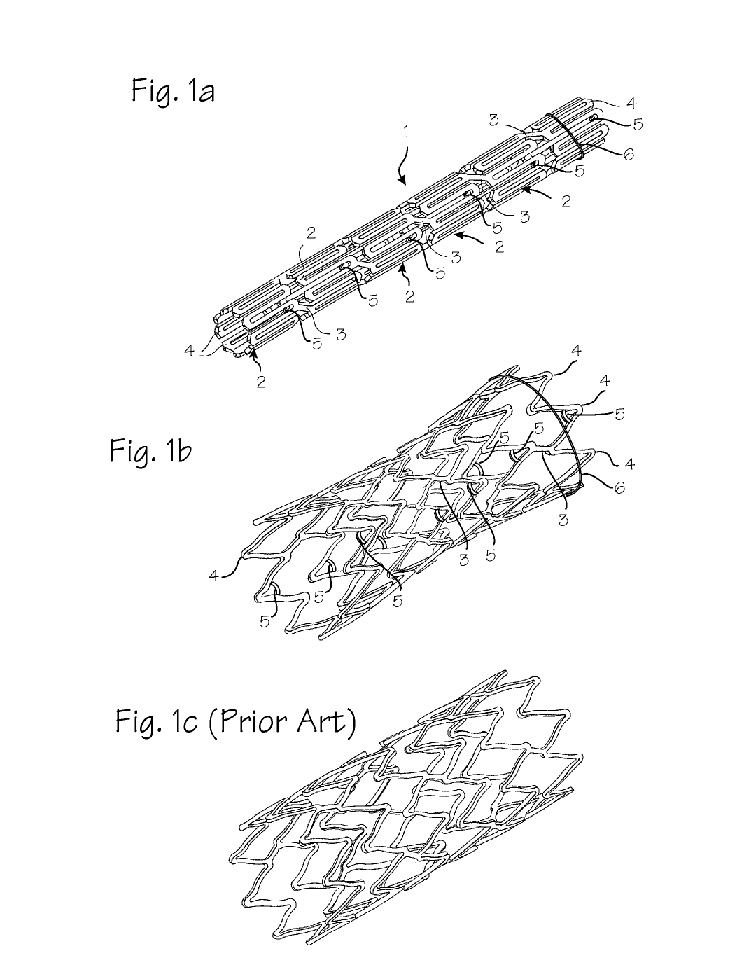

FIGS. 1a through 1c illustrate a stent for use in implementing the therapeutic regimen which includes installation of the stent in a blood vessel (typically a coronary artery), elution of an MMPI from a coating on the stent over the course of 1 to 3 months, and co-incident expansion of the stent and retreat of the vessel wall over 1 to 3 months, at which point the stent expansion is limited by restraints and the elution of MMPI has reached a minimal level. The stent may be made of various self-expanding materials, such as nitinol and other shape-memory or pseudoelastic materials, stainless steel, etc. The stent may be made with various self-expanding constructions, such as a braided, knitted or mesh tube of pseudoelastic material (similar to the Abbot Xact® Carotid Stent or the Boston Scientific Nexstent™ carotid stent), a multi-link stent, a diamond-celled stent, or honeycomb-celled stent. The stent may comprise several zig-zag hoops jointed by struts (similar to the Sirolumis® stent of Boston Scientific or the J&J Cypher® stent, DISA Chromoflex stent, or Antares Starflex stent (though currently only used for renal arteries) the MULTI-LINK VISION® Coronary Stent System from Guidant (which is balloon expandable).

In FIGS. 1a and 1b, the self-expanding stent 1 is shown, with its typical construction of several zig-zag hoop segments 2 joined by various struts 3. The struts may be referred to as connecting bars, connecting links, etc. by various stent manufacturers. The hoop segments may be of quite variable geometry, but generally comprise resiliently or pseudoelastically expanding series of long elements joined to adjacent long elements at alternating joints 4 (also referred to as ends or turns), such that the long elements spread away from each other in scissor-like fashion upon expansion. The restraining means may comprise an inelastic strap 5 connecting circumferentially adjacent struts, thereby limiting the circumferential expansion of these struts relative to each other. The restraining means may comprise one or more inelastic wires 6 circumferentially surrounding the stent, thereby limiting the circumferential expansion of the stent. The restraining means may comprise a physical stop, for those stent geometries permitting it, disposed near a conjunction of stent elements so as to limit the longitudinal approach of one or more longitudinally adjacent struts toward one another along the length of the stent.

The point at which the stent is limited from expanding to its fully unrestrained diameter, in order to achieve the high compliance mismatch, may be determined by a ratio of the stop-restrained diameter to the fully unrestrained diameter. For a coronary artery having a nominal diameter of about 2 mm (and an occluded diameter of 1 to 2 mm), the stent may be constructed and packaged for insertion such that its fully compressed diameter is below 3 mm, its fully unrestrained diameter is 6 mm, and its stop-restrained diameter is 3 to 5 mm. Thus, the stop-restrained diameter is about 50 to 80 percent of the fully unrestrained diameter. At the stop-restrained diameter, the stent will no longer apply force outwardly against a vessel of like diameter, but will resist collapse with significant hoop strength. Thus, the stent of FIG. 1c, which is formed in the same manner as the stent of FIG. 1b except of the absence of any restraining means, opens to a larger diameter when unrestrained than does the stent of FIG. 1b.

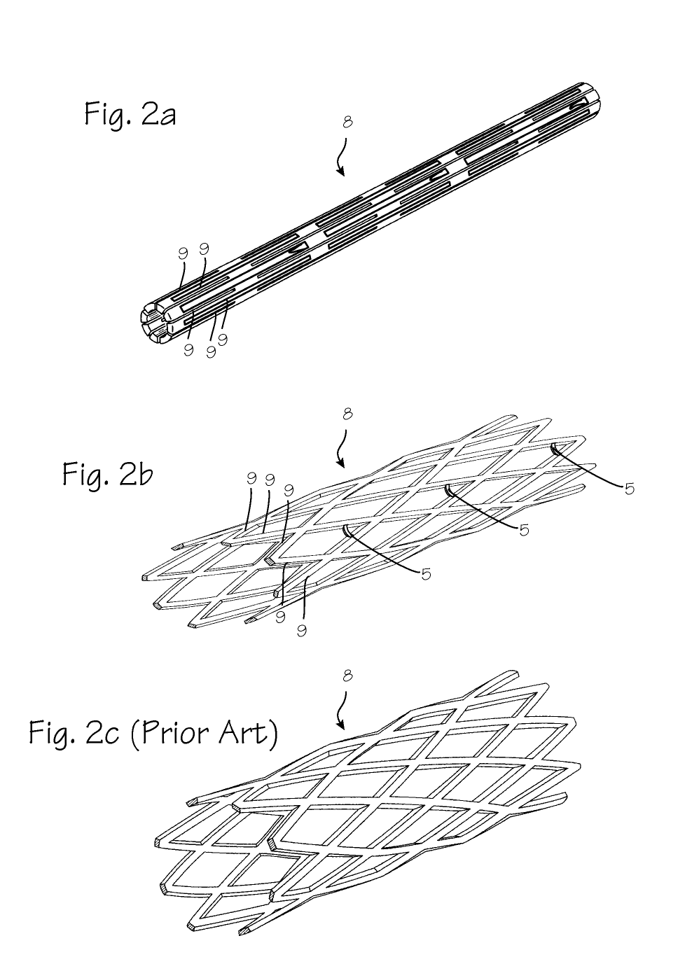

FIGS. 2a and 2b illustrate a stent for use in implementing the therapeutic regimen. This stent 8 is based on the typical diamond-celled design, and includes various stent elements 9, and can be considered analogous to the multi-segment stent of FIGS. 1a through 1c, wherein the segments are directly joined, turn to turn, without the use of the connecting struts. Some adjacent stent elements are joined by restraining means, in the form of straps 5, which limit the expansion of the stent, as shown in FIG. 2b, to a diameter significantly less that the hypothetical unrestrained diameter illustrated in prior art stent of FIG. 2c.

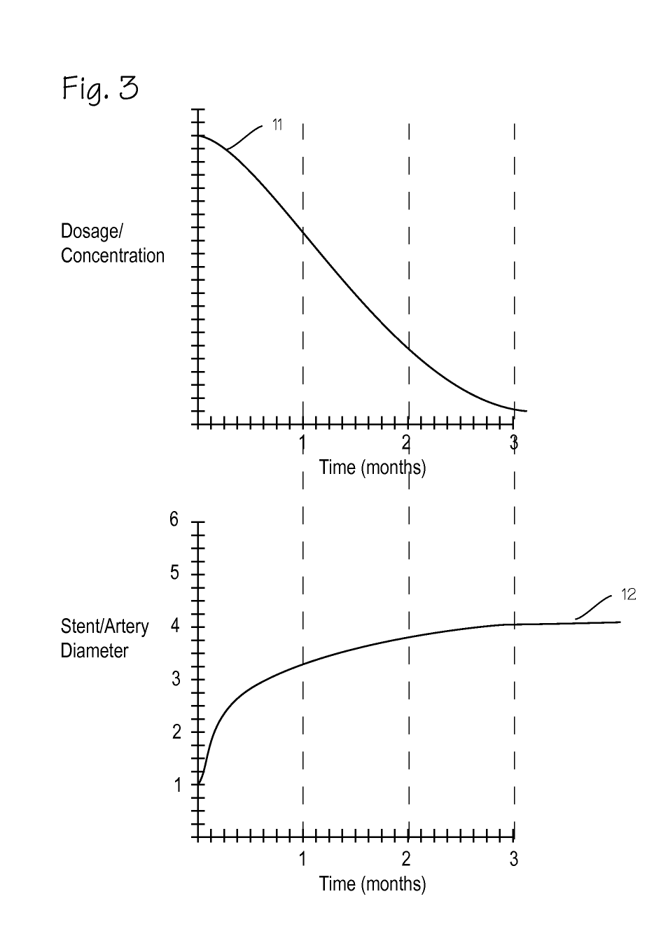

FIG. 3 illustrates the therapeutic regimen to be achieved by the stent described in detail in the preceding paragraphs. As illustrated in the graphs, the dosage delivered by the drug eluting compound decreases over time, while the stent gradually expands to its stop-limited diameter. The initial concentration and/or amount of drug applied to the stent is chosen so that, in a nominal coronary artery, the drug is substantially depleted in a predetermined post-implantation period. The drug is thus eluted from the stent at a therapeutic rate or dosage over this predetermined post implantation period, and depleted such that the amount of drugs eluted after the predetermined post-implantation is negligible or sub-therapeutic. This is shown in the graph of the drug delivery rate, marked as item 11, which approaches negligible levels at the end of the predetermined post-implantation stabilization period. Depletion may be characterized by elution of most of the drug out of the stent coating, or by reduction of the rate of elution to insubstantial levels. The amount and concentration of drugs required depend on the drug, the chosen substrate, and the specific formulation of the drug. Dosage of in the range of 1 to 100 micrograms of drug compound per square mm of stent surface area will be sufficient for most of the compounds proposed herein. While the drug is eluting over the course of the predetermined post-implantation period, the stent is resiliently or pseudoelastically self-expanding and the artery is expanding in response to the stent. This is shown in the graph of the stent diameter/blood vessel diameter marked as item 12, which stabilizes at the end of the predetermined post-implantation stabilization period. The self-expansion is limited by the stops or straps or other limiting means, as discussed in reference to FIGS. 1a through 1c. The compliance mismatch of the stent, having been formulated to present high compliance mismatch on initial placement to force the blood vessel open, lessens as the stent expands further to follow the retreat of the blood vessel, and is finally limited by the limiting means. At this point, the stent will provide sufficient hoop strength to prevent collapse of the blood vessel in response to any natural tendency of the vessel to close, but will not provide additional outward expansive force. The compliance mismatch will be essentially zero, considering compliance mismatch to be any non-zero outward pressure of the stent.

The predetermined post-implantation stabilization period may vary as clinical experience dictates. It may also vary depending on the drug and stent geometry, and the expected physiological response of the particular patient. Currently, a stabilization period of about one to three months is preferred.

Various drugs and compounds may be used in the stents and methods described above. One of the many matrix metalloproteinase inhibitors suggested for use with stents may be used. The terms “matrix metalloproteinase inhibitor” or “MMP inhibitor” or MMPI have been used in the art to refer to numerous compounds including, but not limited, to collagen peptidomimetic and non-peptidomimetic inhibitors, tetracycline and tetracycline derivatives, e.g. hydroxamate peptidomimetic inhibitor batimastat and its orally bio-available analogue marimastat (BB-2516), prinomastat (AG3340), metastat (NSC 683551) BMS-279251, BAY 12-9566, TAA211, MMI270B or AAJ996, trocade, CGS 27023, RS 130830 or AG3340; collagenase, stromelysin, gelatinase and elastase; trocade, CGS 27023, RS 130830 or AG3340. Paclitaxol, rapamycin, Sirolimus™, and other anti-proliferative compounds (and all of their various derivative compounds) currently in use may be used in conjunction with the MMPI.

Various compounds may be used as a carrier or substrate for the eluting drug, such as poly(styrene-b-isobutylene-b-styrene) (used in the Taxus stent), PEVA/PBMA copolymer (used in the CYPHER stent). Bio-degradable polymers such as polyglycolic acid/polylactic acid, polycaprolactone, polyhydroxybutarate valerate, polyorthoester, polyethylenoxide/polybutylene terepthalate, and polyurethane, as well as non-biodegradable polymers such as silicone, and polyethylene terephthalate may be used as well.

In use, the stent may be manufactured as described above, and coated with eluting drugs to achieve the dosage described above, and mounted on an insertion catheter for insertion into the body. The stent may be navigated to the site of an occlusion in the coronary arteries of a patient, and released to expand resiliently within the occlusion. The stent may be forced open with a balloon mounted on the tip of the insertion catheter or an angioplasty balloon to acutely open the occluded vessel. Thereafter, the stent will further expand, resiliently or pseudoelastically, to track any retreat of the blood vessel, but will not expand beyond the diameter permitted by the limiting means. The drug will elute over time, until exhausted at the end of the predetermined stabilization period. The dosage, stabilization period, initial compliance mismatch and final compliance mismatch or stop-limited diameter may be adjusted as clinical experience suggests.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A method of treating an occluded blood vessel in a patient comprising:
providing a self-expanding stent having a fully expanded diameter when completely unrestrained;
providing a restraining means on the stent, said restraining means adapted to restrain the stent from expanding to its fully expanded diameter
coating the stent with a metalloproteinase inhibitors in a formulation which is substantially exhausted within the blood vessel in one to three months;
implanting the stent in an occluded blood vessel of a patient; and
allowing the stent to expand elastically or pseudoelastically inside the blood vessel over the course of one to three months, until limited by the restraining means;
whereby the stent expansion becomes limited by the restraining means contemporaneously with exhaustion of the metalloproteinase inhibitors.

2. The method of claim 1, wherein the coating step comprises coating the stent with a drug eluting compound including one of the following metalloproteinase inhibitors: collagen peptidomimetic and non-peptidomimetic inhibitors, tetracycline and tetracycline derivatives, batimastat, marimastat (BB-2516), prinomastat (AG3340), metastat (NSC 683551) BMS-279251, BAY 12-9566, TAA211, MMI270B or AAJ996, trocade, CGS 27023, RS 130830 or AG3340.

3. The method of claim 1, further comprising the steps of training the stent to open to the fully expanded diameter exceeding the diameter of a target vessel, providing the restraining means to limit the expansion of the stent to a diameter matching a desired final vessel diameter smaller than the fully expanded diameter.

4. The method of claim 1, further comprising the steps of preparing the stent for implantation into a blood vessel of nominal inner diameter of about 2 mm, training the stent to open to the fully expanded diameter of about 4 mm, providing the restraining means to limit the expansion of the stent to a diameter of about 2 mm, and implanting the stent in an occluded vessel with a nominal inner diameter of 2 mm, and allowing the stent to expand within the blood vessel.

5. A device comprising:
a vascular stent comprising a wire mesh tube including a plurality of stent elements (struts, loops, mesh wires, etc.)), comprising a elastic or pseudoelastic material, said stent having an unrestrained diameter;
restraining means, disposed on the stent, for limiting expansion of the stent to a desired final diameter;
a drug eluting coating disposed on the device, said drug eluting coating comprising a therapeutic dose of an MMP inhibitor, wherein said therapeutic dose is chosen so as to be substantially depleted within the body within one to three months after implantation.

6. The device of claim 5 wherein:
the stent is self-expanding stent having generally longitudinally oriented struts trained to resiliently or pseudoelastically abduct from adjacent struts, and the restraining means comprises a stop disposed on one or more of said struts to impede abduction of the struts.

7. The device of claim 5 wherein:
the stent is a self expanding stent having generally longitudinally oriented struts trained to resiliently or pseudoelastically abduct from adjacent struts, and the restraining means comprises a strap secured to adjacent abducting struts so as to prevent full expansion of the stent to its unrestrained diameter.

8. The device of claim 5 wherein:
the restraining means comprises one or more inelastic hoops surrounding the stent so as to prevent full expansion of the stent to its unrestrained diameter.
